(19) United States
(12) Patent Application Publication
(10) Pub. No.: US 2026/0311103 A1
(43) Pub. Date: Oct. 8, 2026

(54) Integrated Nonprofit Business Model for Sustainable Funding and Holistic Post-Cancer Recovery

(71) Applicant: Melanie Elayne Lewis, Houston, TX (US)

(72) Inventor: Melanie Elayne Lewis, Houston, TX (US)

(21) Appl. No.: 19/098,173

(22) Filed: Apr. 2, 2025

(51) Int. Cl. G06Q 10/067; G06Q 10/0631; G16H 10/60; G16H 20/70; G16H 80/00

(52) U.S. Cl. CPC G06Q 10/067; G06Q 10/06312; G16H 10/60; G16H 20/70; G16H 80/00

(57) ABSTRACT

This invention introduces a nonprofit business model that integrates residential services, business operations, and strategic partnerships to create a self-sustaining funding system while supporting post-cancer patients and their families. The model incorporates housing assistance, career development, financial literacy, health and wellness programs, and identity restoration services. Unlike traditional nonprofits, Grace Anchored Inc. generates revenue through retail sales, contractor partnerships, and business collaborations to sustain operations long-term.

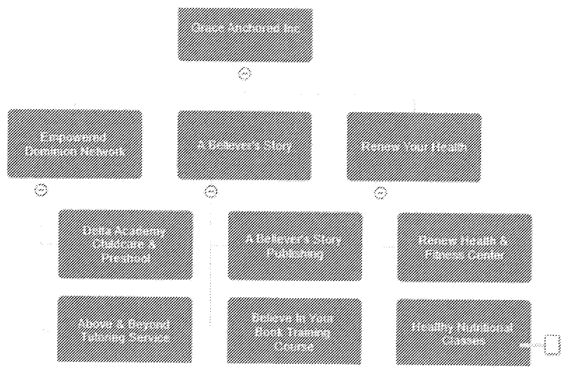

### CROSS-REFERENCE TO RELATED APPLICATIONS

This application does not claim the benefit of any prior U.S. Provisional or Non-Provisional Patent Application.

### BACKGROUND OF THE INVENTION

### 1. Field of the Invention

The present invention relates to a comprehensive nonprofit business model that integrates residential services, economic empowerment, post-cancer recovery programs, and sustainable funding strategies. This model leverages multiple revenue streams, business partnerships, and direct service provision to ensure long-term financial sustainability while supporting post-cancer patients, caregivers, and families.

### 2. Description of Related Art

Traditional nonprofit organizations often rely on donations, grants, and government funding, making financial sustainability a significant challenge. Many cancer recovery programs focus on medical treatment and counseling but lack integrated economic and career support for long-term stability. Grace Anchored Inc. introduces a self-sustaining model that incorporates business operations, contractor partnerships, community engagement, and holistic recovery services to ensure continued impact and financial independence.

### SUMMARY OF THE INVENTION

The invention is a hybrid nonprofit operational model that integrates:
1. Post Cancer Support
   Long-term housing assistance for post-cancer patients and families.
   Family & caregiver support through counseling and educational resources.
2. Health & Wellness Services
   Holistic healing programs, Including nutrition education and fitness training.
   Emotional and spiritual wellness programs through faith-based and holistic counseling.
3. Financial Literacy & Economic Empowerment
   Workforce training, career coaching, and entrepreneurship programs
   Homeownership assistance and financial stability workshops.
4. Business Ventures for Sustainable Funding
   Retail operations, including merchandise and health/wellness product sales.
   Strategic partnerships with for-profit businesses an contractors for funding.
5. Identity Restoration & Personal Rebranding
   Medical tattoo services for post-surgical patients.
   Makeovers, photoshoots, and self-confidence programs.
   Personal book publishing & storytelling projects for patient empowerment.
6. Community Engagement & Advocacy
   Policy advocacy for patient rights and accessibility improvements.
   Public awareness campaigns and mentorship programs for children of post-cancer patients.

### DETAILED DESCRIPTION OF THE INVENTION

### 1. Nonprofit Business Model with For-Profit Integration

The invention establishes a self-sustaining nonprofit by Integrating for-profit b Business models within a nonprofit framework. Instead of relying solely on donations and g an the organization generates revenue through:
Retail sales (merchandise, health & wellness products).
Contractor collaborations to provide housing and post-cancer services.
Business partnerships with for-profit companies to offer services and receive funding.

### 2. Post-Cancer Housing & Stability Services

The nonprofit provides long-term housing to p cancer patients and their families, ensuring safe and stable accommodations while they regain independence. This includes:
Short-term and long-term housing assistance.
Homeownership support & financial literacy training.
Collaboration with local contractors for construction and renovation services.

### 3. Career & Workforce Development

Skilled trade classes through partnership programs.
Resume-building, job training, and employment placement assistance.
Small business development and entrepreneur ship mentorship.

### 4. Personal Restoration & Mental Health Support

Medical tattoo services for patients recovering from surgical procedures.
Makeovers, photoshoots, and confidence-boosting activities.
Book writing and publishing services o document patients' journeys.

### 5. Holistic Health & Wellness Services

Mental health counseling & faith-based spiritual guidance.
Physical wellness programs (nutrition education, fitness, meditation, etc.).
Family and caregiver support services.

### 6. Sustainable Revenue Model

Unlike traditional nonprofits, Grace Anchored Inc. sustains its operation s through diversified funding streams:
Retail sales (merchandise, wellness prod books, etc.).
Strategic partnerships with businesses that fund service operations.
Contractor service agreements for housing and skilled labor partnerships.
Community grants and fundraising programs.

DRAWINGS (If Applicable)

Diagrams illustrating the organizational structure, funding flow, and service integration model can be included as required by USPTO submission guidelines.

### CONCLUSION

This nonprofit business model patent introduces an innovative framework for post-cancer recover and financial sustainability. By integrating business ventures, partnerships, housing support, workforce training, and wellness services, Grace Anchored Inc. ensures long-term stability and empowerment for those affected by cancer.

1. Hybrid Nonprofit-For-Profit Business Model
A nonprofit business model comprising:
(a) a structured framework that integrates for-profit operations within a nonprofit organization;
(b) one or more revenue-generating business ventures selected from the group consisting of retail sales, health and wellness products, and personal branding services;
(c) strategic partnerships with for-profit entities and independent contractors to provide services including but not limited to housing, skilled trades, and counseling; and
(d) a system of reinvestment of earned revenue into nonprofit programming to deliver residential services, wellness support, economic empowerment, and advocacy programs for post-cancer individuals and their families.

2. System Claim
A system for administering transitional support services to post-chemotherapy patients, the system comprising:
(a) a housing infrastructure adapted for immunocompromised individuals;
(b) a digital platform configured to coordinate resource delivery from multiple providers;
(c) a communication interface for patients to access scheduled services and care updates;
(d) a database storing individualized care plans, medical records, and progress notes; and
(e) an analytics engine for evaluating care outcomes and adjusting resource allocations.

3. Method+Apparatus—Combined Claim
An integrated system for post-cancer recovery and reintegration, the system comprising:
(a) a personal empowerment and identity restoration process configured to support emotional, psychological, and social recovery of a post-cancer patient, the process comprising;
(i) administering medical tattoo services to conceal surgical scars and address physical changes;
(ii) conducting professional makeover sessions including hair, wardrobe, and skincare support;
(iii) providing guided photoshoots configured to rebuild patient self-image;
(iv) facilitating personal storytelling and authorship through customized book publishing services; and
(v) delivering mentorship and empowerment workshops to promote long-term emotional healing and social reintegration;
(b) a trauma-informed residential and service apparatus configured to provide physical space and wellness infrastructure for post-cancer care, the apparatus comprising:
(i) at least one modular living unit including medical-grade ventilation, antimicrobial surfaces, and child-friendly accommodations;
(ii) integrated sensory wellness features, including light therapy systems and calming audio systems to support emotional recovery;
(iii) a secure, on-site counseling facility with private access for trauma-informed therapy sessions; and
(iv) at least one multi-room area configured for education, nutrition, and mentorship programming onsite;
wherein the integrated system establishes a novel and exclusive model for post-cancer recovery and reintegration not currently available in the healthcare or nonprofit sectors, and wherein entry into the post-cancer care recovery market through said model requires engagement with the claimed system.
